(19) United States
(12) Patent Application Publication
(10) Pub. No.: US 2024/0225426 A1
(43) Pub. Date: Jul. 11, 2024

(54) FOLDING ENDOSCOPE

(71) Applicant: ScopeAround, Irvine, CA (US)

(72) Inventor: Yingjie SUN, Irvine, CA (US)

(21) Appl. No.: 18/165,656

(22) Filed: Feb. 7, 2023

(30) Foreign Application Priority Data
Jan. 10, 2023 (CN) 202320077112.1

(51) Int. Cl. A61B 1/005; A61B 1/00

(52) U.S. Cl. CPC A61B 1/0051; A61B 1/00128; A61B 1/0016

(57) ABSTRACT

Provided is a folding endoscope, including a head connecting pipe and a tail connecting pipe that is detachably connected through a threaded structure, wherein a connecting line is sleeved between inner cavities of the head connecting pipe and the tail connecting pipe; a camera assembly is mounted at one end, away from the tail connecting pipe, of the head connecting pipe; one end of the connecting line is electrically connected to the camera assembly; an aviation plug is fixedly mounted at one end, away from the head connecting pipe, of the tail connecting pipe; the aviation plug is electrically connected to the connecting line; and a clamping sleeve is slidingly clamped on the head connecting pipe.

### TECHNICAL FIELD

The present invention relates to the technical field of barrel endoscopes, and in particular to a folding endoscope.

### BACKGROUND

Accurate observation of the internal state of a bore is a necessary process to detect the state of a barrel. In the past, the usual method was to illuminate the bore and then look at the bore from front to back, so that the state of the bore could be mastered, but the detailed state could not be clearly understood. With the development of technology, the bore is detected by a barrel endoscope at present. The existing barrel endoscope is long and inconvenient to store and carry, is blurred when looking at an object near a focus of a camera, and is difficult to control and focus. For the above problems, the inventor proposed a folding endoscope to solve the above problems.

### SUMMARY

To solve the problems that a bore is detected by a barrel endoscope at present, and the existing barrel endoscope is long and inconvenient to store and carry, is blurred when looking at an object near a focus of a camera, and is difficult to control and focus, an objective of the present invention is to provide a folding endoscope.

To solve the above technical problems, the present invention adopts the following technical solutions: a folding endoscope includes a head connecting pipe and a tail connecting pipe, wherein one end of the head connecting pipe is detachably connected to one end of the tail connecting pipe through a threaded structure; a connecting line is sleeved between inner cavities of the head connecting pipe and the tail connecting pipe; a camera assembly is mounted at one end, away from the tail connecting pipe, of the head connecting pipe; one end of the connecting line is electrically connected to the camera assembly; an aviation plug is fixedly mounted at one end, away from the head connecting pipe, of the tail connecting pipe; the aviation plug is electrically connected to the other end of the connecting line; and a clamping sleeve is slidingly clamped on the head connecting pipe.

Preferably, the camera assembly includes a connecting ring; one end of the connecting ring is in threaded connection with the head connecting pipe, and the other end of the connecting ring is in threaded connection with one end of a guide pipe; a rotating ring is rotatably clamped at the other end of the guide pipe; a camera lens is mounted in an inner cavity of the rotating ring; an image sensor is slidingly clamped in the guide pipe; the bottom of the rotating ring is fixedly connected to a spiral spring; and the spiral spring is sleeved in the image sensor in a threaded manner.

Preferably, outer walls of the guide pipe and the rotating ring are provided with knurls; an inner cavity of the guide pipe is provided with a guide groove; the guide groove is of a cross structure; and the image sensor is slidingly clamped at the guide groove.

Preferably, the tail connecting pipe includes a splicing pipe, a holding pipe and a tail pipe; one end of the splicing pipe is in threaded connection with one end of the holding pipe; the other end of the holding pipe is in threaded connection with the tail pipe; a threaded head is integrally formed at one end, away from the holding pipe, of the splicing pipe; the threaded head is in thread fit with one end, away from the camera assembly, of the head connecting pipe; and one end, away from the holding pipe, of the tail pipe is fixedly connected to the aviation plug.

Preferably, an outer diameter of each of the splicing pipe, the holding pipe and the tail pipe is equal to an outer diameter of the head connecting pipe; outer walls of the head connecting pipe, the splicing pipe and the holding pipe are provided with antiskid stripes; an inner pipe is fixedly sleeved at an inner wall of the holding pipe; a switch is mounted in the inner pipe; a through groove is formed in the inner pipe and the holding pipe; a toggling block is mounted in the through groove; and the toggling block is connected to the switch.

Preferably, the clamping sleeve includes a sliding sleeve; the sliding sleeve is slidingly sleeve on the head connecting pipe; a friction ring is fixedly mounted at one end of the sliding sleeve; a clamping ring with an elastic plastic C-shaped structure is integrally formed on an outer wall of the sliding sleeve; and the clamping ring is matched with the tail connecting pipe.

Compared with the prior art, the present invention has the following beneficial effects:

1, the threaded head facilitates disassembling and assembling of the tail connecting pipe and the head connecting pipe; the connecting line penetrates through a middle part; when not in use, the tail connecting pipe is separated from the head connecting pipe and is bent through the exposed connecting line, and the tail connecting pipe is clamped in the clamping ring to realize folding and carrying;
2, after unfolding, the threaded head is connected to the head connecting pipe; rotation corresponds to the rotating ring; the rotating ring drives the spiral spring to rotate; the spiral spring drives the image sensor in a threaded manner to move to focus; and the sliding sleeve can slide from the head connecting pipe to the holding pipe, which is convenient for the head connecting pipe to extend into the bore for observation, thereby facilitating use.

### BRIEF DESCRIPTION OF THE DRAWINGS

The drawings of the specification that constitutes a part of the present application are used to provide a further understanding of the present invention. The exemplary embodiments of the present invention and the description thereof are used to explain the present invention, and do not constitute an improper limitation of the present invention. In the drawings:

FIG. 1 is a structural schematic diagram of an embodiment;

FIG. 2 is an exploded view of a structure according to an embodiment;

FIG. 3 is a schematic diagram of an enlarged structure at a position A in FIG. 2;

FIG. 4 is a schematic diagram of an enlarged structure at a position C in FIG. 3;

FIG. 5 is a schematic diagram of a structure at a guide ring according to an embodiment;

FIG. 6 is a schematic diagram of an enlarged structure at a position B in FIG. 2;

FIG. 7 is a schematic diagram of a structure at an inner pipe according to an embodiment; and

FIG. 8 is a schematic diagram of an unfolded structure during use of an embodiment.

Description of Reference Numerals:

1, 1. head connecting pipe; 2. camera assembly; 21. connecting ring; 22. spiral spring; 23. image sensor; 24. guide pipe; 241. guide groove; 25. rotating ring; 26. camera lens; 3. tail connecting pipe; 31. splicing pipe; 32. holding pipe; 321. inner pipe; 33. tail pipe; 34. threaded head; 35. toggling block; 36. through groove; 4. connecting line; 5. aviation plug; 6. clamping sleeve; 61. friction ring; 62. sliding sleeve; 63. clamping ring.

### DETAILED DESCRIPTION OF THE EMBODIMENTS

The present invention is described in detail below with reference to the accompanying drawings and embodiments. Each example is provided to explain the present invention instead of limiting the present invention. In fact, those skilled in the art will appreciate that modifications and variations may be made in the present invention without departing from the scope or spirit of the present invention. For example, features shown or described as one part of one embodiment may be applied to another embodiment to generate yet another embodiment. Therefore, it is expected that the present invention includes such modifications and variations that fall within the scope of the appended claims and their equivalents.

In the description of the present invention, orientation or position relationships indicated by terms “longitudinal”, “transverse”, “upper”, “lower”, “front”, “rear”, “left”, “right”, “vertical”, “horizontal”, “top”, “bottom” and the like are orientation or position relationships shown in the drawings, and these terms are merely for facilitating description of the present invention, but not for requiring that the present invention must be constructed and operated in a specific orientation, and thus, these terms cannot be understood as a limitation to the present invention. As used in the present invention, the terms “connected”, “connection” and “set” should be understood in a broad sense, for example, they may be fixed connection or detachable connection, they may be direct connection or indirect connection through an intermediate part; or they may be wired connection and wireless connection, or may be connection through a wireless communication signal. For those of ordinary skill in the field, the specific meanings of the terms may be understood according to the specific conditions.

The accompanying drawings show one or a plurality of examples of the present invention. The detailed description uses reference numerals and letters to refer to the features in the accompanying drawings. Similar numeral references in the drawings and description have been used to refer to the similar parts in the present invention. As used herein, the terms “first”, “second” and “third” are used interchangeably to distinguish one component from another component, and are not intended to indicate the position or importance of individual components.

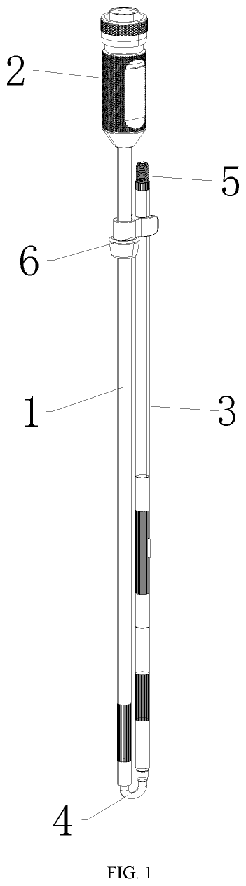

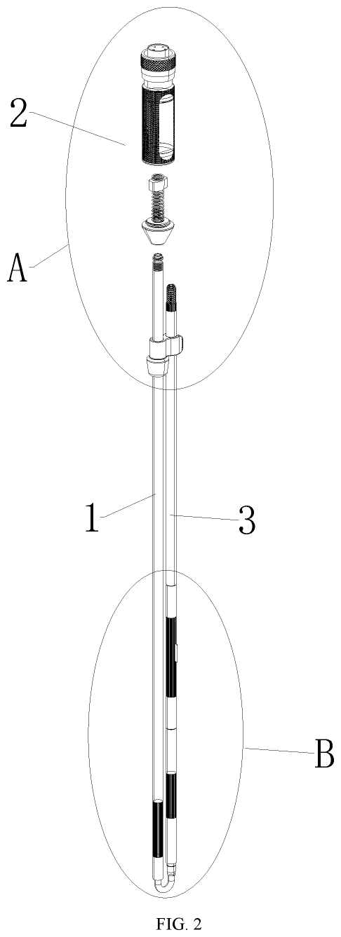

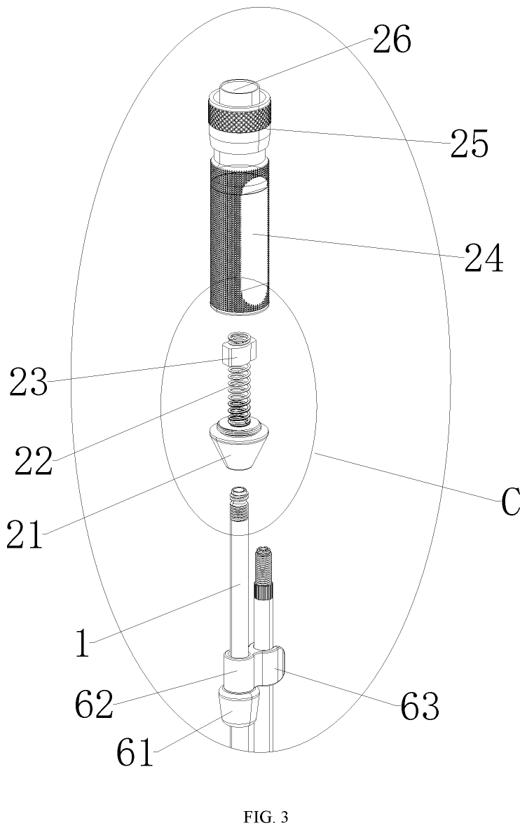

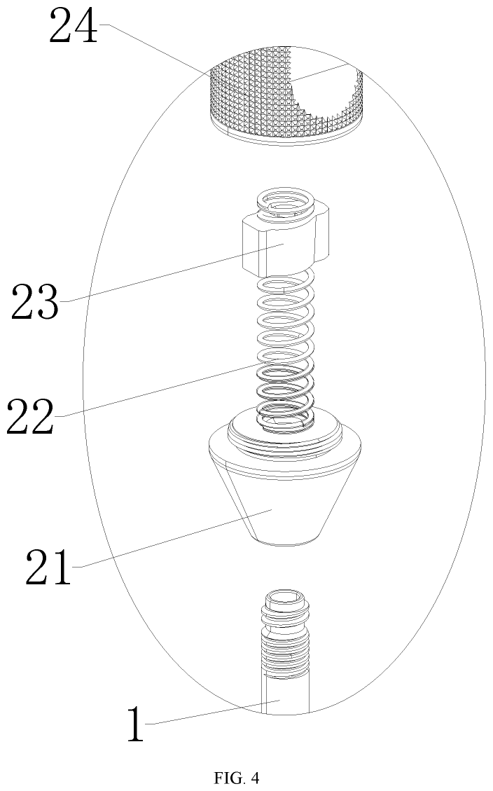

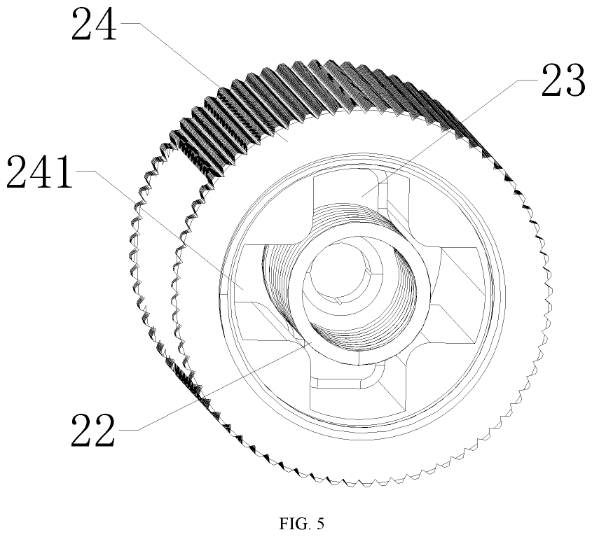

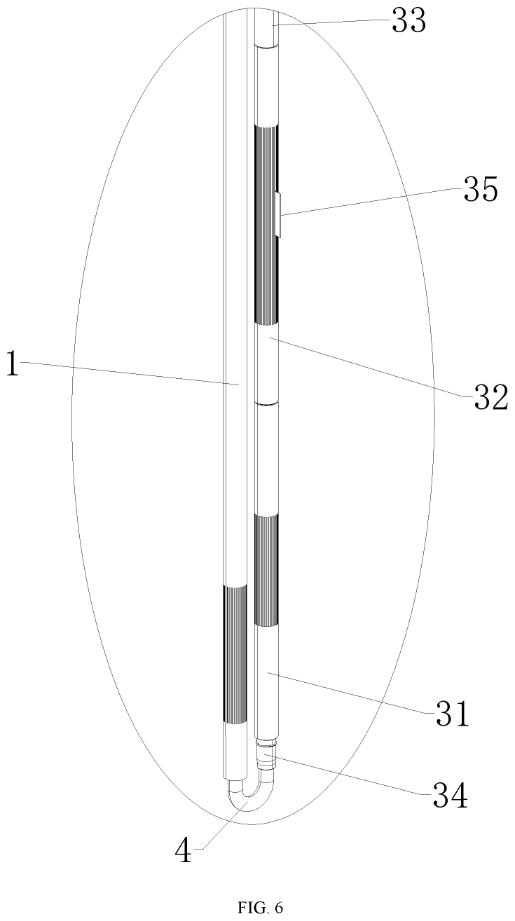

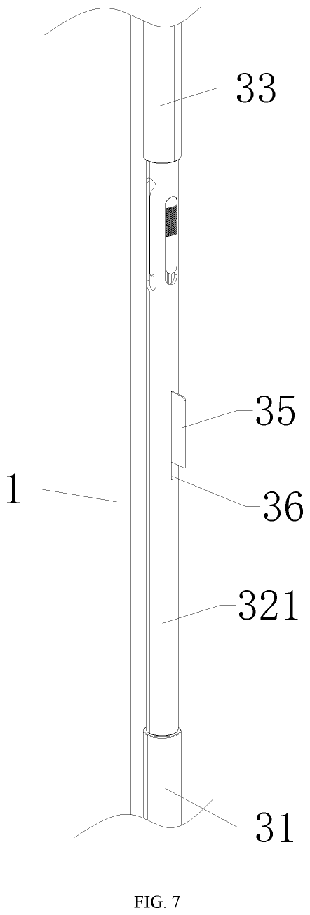

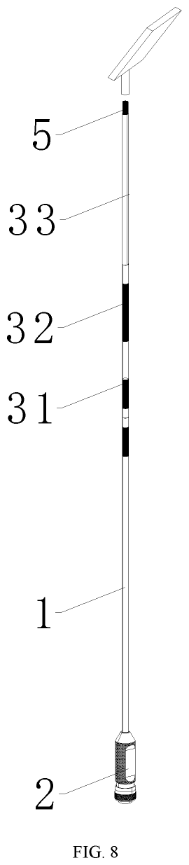

Embodiments: as shown in FIG. 1 to FIG. 8, the present invention provides a folding endoscope, including a head connecting pipe 1 and a tail connecting pipe 3, wherein one end of the head connecting pipe 1 is detachably connected to one end of the tail connecting pipe 3 through a threaded structure; a connecting line 4 is sleeved between inner cavities of the head connecting pipe 1 and the tail connecting pipe 3; a camera assembly 2 is mounted at one end, away from the tail connecting pipe 3, of the head connecting pipe 1; one end of the connecting line 4 is electrically connected to the camera assembly 2; an aviation plug 5 is fixedly mounted at one end, away from the head connecting pipe 1, of the tail connecting pipe 3; the aviation plug 5 is electrically connected to the connecting line 4; and a clamping sleeve 6 is slidingly clamped on the head connecting pipe 1.

By the above technical solution, the tail connecting pipe 3 and the head connecting pipe 1 are detachably connected through the threaded structure, the connecting line 4 penetrates through a middle part; when not in use, the tail connecting pipe 3 is separated from the head connecting pipe 1 and is bent through the exposed connecting line 4, so that the tail connecting pipe 3 and the head connecting pipe 1 are folded and are clamped through a clamping sleeve 6, thereby facilitating storage and carrying; and during use, the connecting line 4 is completely inserted into the head connecting pipe 1 and the tail connecting pipe 3, and the tail connecting pipe 3 and the head connecting pipe 1 are in threaded connection to form a rod body which extends into the bore through the camera assembly 2, thereby facilitating focusing observation and use.

Further, the camera assembly 2 includes a connecting ring 21; one end of the connecting ring 21 is in threaded connection with the head connecting pipe 1, and the other end of the connecting ring 21 is in threaded connection with one end of the guide pipe 24; a rotating ring 25 is rotatably clamped at the other end of the guide pipe 24; a camera lens 26 is mounted on an inner cavity of the rotating ring 25; an image sensor 23 is slidingly clamped in the guide pipe 24; the bottom of the rotating ring 25 is fixedly connected to a spiral spring 22; the spiral spring 22 is sleeved in the image sensor 23 in a threaded manner; outer walls of the guide pipe 24 and the rotating ring 25 are provided with knurls; an inner cavity of the guide pipe 24 is provided with a guide groove 241; the guide groove 241 is of a cross structure; and the image sensor 23 is slidingly clamped at the guide groove 241.

By the above technical solution, rotation corresponds to the rotating ring 25; the rotating ring 25 drives the spiral spring 22 to rotate; the spiral spring 22 drives the image sensor 23 in a threaded manner to move to focus; and the inner cavity of the spiral spring 22 facilitates the penetration of the connecting line 4 to electrically connect the camera lens 26 and the image sensor 23.

Further, the tail connecting pipe 3 includes a splicing pipe 31, a holding pipe 32 and a tail pipe 33; one end of the splicing pipe 31 is in threaded connection with one end of the holding pipe 32; the other end of the holding pipe 32 is in threaded connection with the tail pipe 33; a threaded head 34 is integrally formed at one end, away from the holding pipe 32, of the splicing pipe 31; the threaded head 34 is in thread fit with one end, away from the camera assembly 2, of the head connecting pipe 1; and one end, away from the holding pipe 32, of the tail pipe 33 is fixedly connected to the aviation plug 5.

Further, an outer diameter of each of the splicing pipe 31, the holding pipe 32 and the tail pipe 33 is equal to an outer diameter of the head connecting pipe 1; outer walls of the head connecting pipe 1, the splicing pipe 31 and the holding pipe 32 are provided with antiskid stripes; an inner pipe 321 is fixedly sleeved at an inner wall of the holding pipe 32; a switch is mounted in the inner pipe 321; a through groove 36 is formed in the inner pipe 321 and the holding pipe 32; a toggling block 35 is mounted in the through groove 36; and the toggling block 35 is connected to the switch.

By the above technical solution, the threaded head 34 facilitates disassembling and assembling of the tail connecting pipe 3 and the head connecting pipe 1; the toggling block 35 at the holding pipe 32 is used to control the switch so as to adjust the focal length of the camera lens; the aviation plug 5 is connected to an external image processing/display device and a power supply; and a 45° mirror may be mounted and is fed to a peeping place to reflect an image.

Further, the clamping sleeve 6 includes a sliding sleeve 62; the sliding sleeve 62 is slidingly sleeved on the head connecting pipe 1; a friction ring 61 is fixedly mounted at one end of the sliding sleeve 62; a clamping ring 63 with an elastic plastic C-shaped structure is integrally formed on an outer wall of the sliding sleeve 62; and the clamping ring 63 is matched with the tail connecting pipe 3.

By the above technical solution, during folding, the tail connecting pipe 3 is clamped in the clamping ring 63 to realize folding and carrying; and after unfolding, the sliding sleeve 62 can slide from the head connecting pipe 1 to the holding pipe 32, which is convenient for the head connecting pipe 1 to extend into the bore.

The working principle: the threaded head 34 facilitates disassembling and assembling of the tail connecting pipe 3 and the head connecting pipe 1; the connecting line 4 penetrates through a middle part; when not in use, the tail connecting pipe 3 is separated from the head connecting pipe 1 and is bent through the exposed connecting line 4, and the tail connecting pipe 3 is clamped in the clamping ring 63 to realize folding and carrying; after unfolding, the threaded head 34 is connected to the head connecting pipe 1; rotation corresponds to the rotating ring 25; the rotating ring 25 drives the spiral spring 22 to rotate; the spiral spring 22 drives the image sensor 23 in a threaded manner to move to focus; and the sliding sleeve 62 can slide from the head connecting pipe 1 to the holding pipe 32, which is convenient for the head connecting pipe 1 to extend into the bore for observation, thereby facilitating use.

It will be apparent to those skilled in the art that various modifications and variations of the present invention can be made without departing from the spirit or scope of the present invention. Therefore, the present invention is intended to cover these modifications and variations provided that they fall within the scope of the claims of the present invention and their equivalent technologies.

1. A folding endoscope, comprising a head connecting pipe and a tail connecting pipe, wherein one end of the head connecting pipe is detachably connected to one end of the tail connecting pipe through a threaded structure; a connecting line is sleeved between inner cavities of the head connecting pipe and the tail connecting pipe; a camera assembly is mounted at one end, away from the tail connecting pipe, of the head connecting pipe; one end of the connecting line is electrically connected to the camera assembly; an aviation plug is fixedly mounted at one end, away from the head connecting pipe, of the tail connecting pipe; the aviation plug is electrically connected to the other end of the connecting line; and a clamping sleeve is slidingly clamped on the head connecting pipe.

2. The folding endoscope according to claim 1, wherein the camera assembly comprises a connecting ring; one end of the connecting ring is in threaded connection with the head connecting pipe, and the other end of the connecting ring is in threaded connection with one end of a guide pipe; a rotating ring is rotatably clamped at the other end of the guide pipe; a camera lens is mounted in an inner cavity of the rotating ring; an image sensor is slidingly clamped in the guide pipe; the bottom of the rotating ring is fixedly connected to a spiral spring; and the spiral spring is sleeved in the image sensor in a threaded manner.

3. The folding endoscope according to claim 2, wherein outer walls of the guide pipe and the rotating ring are provided with knurls; an inner cavity of the guide pipe is provided with a guide groove; the guide groove is of a cross structure; and the image sensor is slidingly clamped at the guide groove.

4. The folding endoscope according to claim 1, wherein the tail connecting pipe comprises a splicing pipe, a holding pipe and a tail pipe; one end of the splicing pipe is in threaded connection with one end of the holding pipe; the other end of the holding pipe is in threaded connection with the tail pipe; a threaded head is integrally formed at one end, away from the holding pipe, of the splicing pipe; the threaded head is in thread fit with one end, away from the camera assembly, of the head connecting pipe; and one end, away from the holding pipe, of the tail pipe is fixedly connected to the aviation plug.

5. The folding endoscope according to claim 4, wherein an outer diameter of each of the splicing pipe, the holding pipe and the tail pipe is equal to an outer diameter of the head connecting pipe; outer walls of the head connecting pipe, the splicing pipe and the holding pipe are provided with antiskid stripes; an inner pipe is fixedly sleeved at an inner wall of the holding pipe; a switch is mounted in the inner pipe; a through groove is formed in the inner pipe and the holding pipe; a toggling block is mounted in the through groove; and the toggling block is connected to the switch.

6. The folding endoscope according to claim 1, wherein the clamping sleeve comprises a sliding sleeve; the sliding sleeve is slidingly sleeve on the head connecting pipe; a friction ring is fixedly mounted at one end of the sliding sleeve; a clamping ring with an elastic plastic C-shaped structure is integrally formed on an outer wall of the sliding sleeve; and the clamping ring is matched with the tail connecting pipe.
